(19) United States
(12) Patent Application Publication
(10) Pub. No.: US 2016/0175482 A1
(43) Pub. Date: Jun. 23, 2016

(54) COMPOSITION AND DELIVERY SYSTEM

(71) Applicant: LOCATE THERAPEUTICS LIMITED, Nottingham (GB)

(72) Inventors: Robin Andrew QUIRK, Nottinghamshire (GB); Helen Celia COX, Nottinghamshire (GB); Kevin Morris SHAKESHEFF, Nottinghamshire (GB)

(73) Assignee: LOCATE THERAPEUTICS LIMITED, Nottingham (GB)

(21) Appl. No.: 14/910,436

(22) PCT Filed: Aug. 8, 2014
(86) PCT No.: PCT/GB2014/052438

§ 371 Date: Feb. 5, 2016

(30) Foreign Application Priority Data
Aug. 9, 2013 (GB) 1314312.8

(51) Int. Cl. A61L 27/18; A61L 27/56; A61L 27/54

(52) U.S. Cl. CPC A61L 27/18; A61L 27/54; A61L 27/56; A61L 2300/216

(57) ABSTRACT

An injectable agent delivery system comprising a composition that comprises: an agent for sustained delivery located within discrete particles; and an injectable scaffold material comprising discrete particles which are capable of interacting to form a scaffold, and uses thereof.

The invention relates to injectable scaffolds, and to the use of such scaffolds in delivery systems to deliver an agent to a target site in a subject.

Within the field of regenerative medicine there are many opportunities for new clinical procedures that stimulate tissue repair by localising agents, such as growth factors or cells at a specific location within the patient. Examples of clinical opportunities include regeneration of cardiac muscle after an infarction, induction of bone growth in spinal fusion, healing of diabetic foot ulcers and limitation or, perhaps, reversal of damage due to stroke. The localisation of agents, such as growth factors, can be achieved using scaffolds. Scaffolds provide an appropriate mechanical environment, architecture and surface chemistry for angiogenesis and tissue formation. The use of scaffolds as drug or cell delivery systems has great potential but is also very challenging due to the need to tailor the porosity, strength and degradation kinetics of the scaffolds to the tissue type whilst achieving the appropriate kinetics of release of agents, such as proteins that act as growth factors or cells.

A further complication in the use of scaffolds as delivery systems for in vivo repair and/or regeneration is the issue of the route of administration. In many clinical examples the site of tissue requiring repair is either difficult to access (e.g. within the brain for stroke therapies or cardiac muscle for post infarction treatment) or of unknown size and shape. Therefore, there is a need for improved injectable scaffolds that can be administered via minimally invasive procedures.

In broad terms, a scaffold is typically either a pre-formed water-insoluble matrix, with large interconnected pores or a hydrogel. Such scaffolds are implanted into a patient for augmented in vivo tissue repair and/or regeneration.

In terms of implantation, the pre-formed water-insoluble matrices must be shaped to fill a cavity within the body, requiring knowledge of the cavity dimensions and limiting the shape of cavity that can be filled. In addition, an invasive operation is required to deliver the scaffold.

In contrast, a number of hydrogel materials have been designed that can be delivered directly into the body through a syringe. The gel forms within the body following a trigger signal, for example a temperature change or UV light exposure. Such systems have the advantage that they can fill cavities of any shape without prior knowledge of the cavity dimensions. However, such hydrogels lack large interconnected porous networks and, hence, release of an agent from the gel is limited by poor diffusion properties.

Furthermore, the poor mechanical strength of hydrogels means they are often unable to withstand the compressive forces applied in use, furthermore this can result in undesirable delivery properties, as agents in the gels can be in effect squeezed out of the hydrogel.

WO2010/100506 (the contents of which are incorporated herein by reference) provided an injectable, agent delivery system comprising a composition comprising: (i) an injectable scaffold material comprising discrete particles; and (ii) a carrier comprising an agent for delivery. The discrete particles are capable of interacting to form a scaffold.

One use of scaffolds for delivery of agents is in the field of bone repair, in particular in spinal fusion, non-union fractures and dental bone repair. Statin drugs have been shown to promote bone formation through indirect promotion of endogenous bone morphogenetic protein-2 (BMP-2; an osteoinductive growth factor) activity, and stimulation of vascular endothelial growth factor production (VEGF; promotes osteoblast differentiation and blood vessel formation into developing tissues). Statins have a long history of clinical use as oral treatments for hypercholesterolemia. Their metabolism within the body is such that local delivery is indicated for orthopaedic applications.

The concept of local delivery of statins was first described by Mundy et al in 1996 (U.S. Pat. No. 6,022,887, the content of which is hereby incorporated in its entirety). Subsequently, there has been a raft of research papers and preclinical studies of efficacy, but no commercial outputs to date.

The research literature describes the need for an appropriate carrier for local delivery of statins. The ideal material would localise the drug at the site of action, provide a sustained release of the active at a level that stimulates repair without inducing inflammation or other side effects, and ideally would also provide support structure for the infiltration of bone progenitor cells and tissue construction. Obtaining the desired release characteristics in particular has proven challenging and hampered development. Only recently has the replication of in vitro bench data been attempted through rational design of the delivery matrix (Rashidi et al, Polymers, 2010, 2, 709-718).

The inventors have surprisingly found that improved results can be obtained using the delivery system of the current invention. In a first aspect, the invention provides an injectable agent delivery system comprising a composition that comprises an agent for delivery located within discrete particles and an injectable scaffold material comprising discrete particles which are capable of interacting to form a scaffold.

Unlike with many delivery systems relying on retarded diffusion, drug release in the present invention is precisely controlled by material degradation. Sustained release profiles can be varied between weeks to months, and the formulation has a suppressed initial ‘burst’ effect (otherwise typical of depot release systems). The versatility of the system therefore provides a distinct advantage when developing formulations for a drug with a narrow therapeutic index. The unique mechanical and porous properties of the material assist in new tissue and blood vessel formation, acting as a substrate upon which the host can rebuild new structural and functional elements. The invention is therefore able to provide improved release and potency retention when compared to conventional substrates such as collagen, which delivers a short-lived bolus.

The composition of the invention possesses the advantages that it can be used to generate porous scaffolds that self-assemble at the site of injection and which contain an agent and allow the controlled release of the agent at the site of the scaffold formation. By providing enhanced control of release of the agent unwanted effects can be effectively avoided. For example, in the delivery of statins for bone repair, in some prior art delivery systems an initial burst of drug release can result in too much statin being released, risking an unwanted inflammatory response which can block bone formation. The delivery systems, compositions and methods of the invention avoid this problem by providing a slower and more controlled retarded release profile.

In some embodiments the agent for delivery is located within the discrete particles which are capable of interacting to form a scaffold. In some embodiments, the composition of the delivery system includes discrete particles in addition to those that are capable of interacting to form a scaffold, and in such embodiments the agent for delivery may be located within those discrete particles which are not those capable of interacting to form a scaffold or in those discrete particles which are capable of interacting to form a scaffold. In some embodiments having particles which can and particles which cannot interact to form a scaffold, an agent for delivery is located in both types of particle.

The invention further provides a composition comprising an agent for delivery located within discrete particles and an injectable scaffold material comprising discrete particles which are capable of interacting to form a scaffold, for use in a method of treatment of the human or animal body by surgery or therapy or in a diagnostic method practised on the human or animal body. Preferably the composition is for pharmaceutical use or cosmetic surgery.

The composition may be a composition for use in a method of treatment or prevention of a condition selected from: neurodegeneration disorders (e.g. post stroke, Huntington's, Alzheimer's disease, Parkinson's disease), bone-related disorders (including osteoarthritis, spinal disk atrophy, bone cavities requiring filling, bone fractures requiring regeneration or repair), burns, cancers, liver disorders (including hepatic atrophy), kidney disorders (including atrophy of the kidney), disorders of the bladder, ureter or urethra (including damaged ureter or damaged bladder requiring reconstruction, prolapse of the bladder or the uterus), diabetes mellitus, infertility requiring IVF treatment, muscle wasting disorders (including muscular dystrophy), cardiac disorders (e.g. damaged cardiac tissue post myocardial infarction, congestive heart disease), eye disorders (e.g. damaged or diseased cornea), damaged vasculature requiring regeneration or repair, ulcers, and damaged tissue requiring regeneration or reconstruction (including damaged organ requiring regeneration or reconstruction, and damaged nerves requiring regeneration or reconstruction).

Preferably the agent may be a therapeutically, prophylactically or diagnostically active substance. It may be any bioactive agent. The agent for delivery may be a drug, a cell, signalling molecule, such as a growth factor, or any other suitable agent.

For example, the agent may comprise amino acids, peptides, proteins, sugars, antibodies, nucleic acid, antibiotics, antimycotics, growth factors, nutrients, enzymes, hormones, steroids, synthetic material, adhesion molecules, colourants/dyes (which may be used for identification), radioisotopes (which may be for X-ray detection and/or monitoring of degradation), and other suitable constituents, or combinations thereof.

It is possible to use any animal cell with the composition of the invention. Examples of cells which may be used include bone, osteoprogenitor cells, cartilage, muscle, liver, kidney, skin, endothelial, gut, intestinal, cardiovascular, cardiomycotes, chondrocyte, pulmonary, placental, amnionic, chorionic, foetal or stem cells. Where stem cells are used, preferably non-embryonic stem cells are used. The cells may be included for delivery to the site of scaffold formation, or they may be included and intended to be retained in the scaffold, for example, to encourage colonisation of the scaffold.

Other agents which may be added include but are not limited to epidermal growth factor, platelet derived growth factor, basic fibroblast growth factor, vascular endothelial growth factor, insulin-like growth factor, nerve growth factor, hepatocyte growth factor, transforming growth factors and other bone morphogenic proteins, cytokines including interferons, interleukins, monocyte chemotactic protein-1 (MCP-1), oestrogen, testosterone, kinases, chemokinases, glucose or other sugars, amino acids, calcification factors, dopamine, amine-rich oligopeptides, such as heparin binding domains found in adhesion proteins such as fibronectin and laminin, other amines, tamoxifen, cis-platin, peptides and certain toxoids. Additionally, drugs (including statins and NSAIDs), hormones, enzymes, nutrients or other therapeutic agents or factors or mixtures thereof may be included.

In some embodiments, the agent for delivery is a statin, e.g. simvastatin, atorvastatin, fluvastatin, pravastatin or rosuvastatin. Preferably the statin is simvastatin. Embodiments in which the agent is a statin are particularly suitable for the treatment of orthopaedic indications, craniomaxillofacial surgery and dentistry. In some embodiments the treatment is dental bone repair, such as dental ridge restoration. In other embodiments the treatment is the repair of non-union fractures. In other embodiments the treatment is spinal fusion.

Dental bone graft substitutes are primarily used in implant procedures requiring additional bone support. Bone regeneration is enhanced with advanced products, allowing dental bone grafting procedures to be performed on patients who would otherwise not be able to receive such treatment. In approximately 40% of all dental implant cases, there is not enough bone to ensure proper implant integration, and bone graft substitutes are required. Tooth extraction can result in deterioration of alveolar bone, resulting in a chronic progressive condition termed residual ridge resorption (RRR). Standard bone grafting options result in secondary lesions, immunologic rejection and poor long-term outcomes (Wu et al. Int. J. Oral Maxillofac. Surg, 2008, 37, 170-176). Osteoinductive factors released from a non-immunogenic delivery system could provide an answer.

Growth in the dental market will be driven by a variety of factors, including the increased use of bone graft material both in dental implant and periodontal procedures, product improvements, the increased exposure of bone graft products, and an aging global population. Grafting techniques are making it possible to expand the candidate pool for implants to include a sizable population of edentulous patients who were poor candidates for dental implantation due to severe bone resorption. World sales of dental bone grafts reached $130 million in 2006, up 12% over 2005. This was expected to more than double by 2012, with revenues reaching $266 million (http://www.prleap.com/pr/77509/). The U.S. market for dental bone graft substitutes and other biomaterials is expected to grow at a CAGR of 12% over 2012-2018 (http://www.prnewswire.com/news-releases/us-market-for-dental-bone-graftsubstitutes-dental-membranes-and-tissue-engineering-products-2012-137656403.html)

The incidence of long bone fractures in the Western world is between 300-400 individuals per 100,000 per year. Between 5-30% of patients will develop complications during the healing process, leading to delayed union or even non-union of the fracture (Lissenberg-Thunnissen et al, Int Orthop. 2011 Vol. 35 (9), 1271-1280). These complications may induce prolonged hospitalisation and secondary interventions with concomitant inconveniences and costs. Especially for those patients, but also for all patients with fractures, treatments that positively influence bone healing and subsequently shorten the time necessary for bone union are of great interest. Surgical intervention in non-unions is required to re-expose living tissue and to insert an osteoinductive graft material. Using autograft or allograft material, this treatment is successful in 70-80% of cases and costs an estimated $14,000 per patient. There is therefore much interest in more effective graft materials.

Spinal fusion is used to surgically treat vertebral abnormalities such as spinal curvatures (scoliosis or kyphosis), slipped discs (following discectomy), or fractures. The procedure uses graft materials (with or without pedicle screws, plates or cages) or other devices to fuse vertebrae together.

Of the more than one million bone grafts performed worldwide annually, 50% involve spinal fusions, and of these patients, 25% complain of donor site pain from the autograft harvest for up to 2 years postoperatively (Olabisi et al, Spine J. 2011, Vol. 11 (6), 545-556). These complications have driven the search for and subsequent use of alternatives. The invention provides such alternatives in the form of the systems, compositions and methods described herein.

In some embodiments the particles are provided in a carrier. The carrier is preferably an aqueous carrier, in particular water or an aqueous solution or suspension, such as saline, plasma, bone marrow aspirate, buffers, such as Hank's Buffered Salt Solution (HBSS), HEPES (4-(2-hydroxyethyl)-1-piperazineethanesulfonic acid), Ringers buffer, Krebs buffer, Dulbecco's PBS, and normal PBS; simulated body fluids, plasma platelet concentrate and tissue culture medium.

The carrier may, optionally, contain one or more suspending agent. The suspending agent may be selected from carboxy methylcellulose (CMC), mannitol, polysorbate, poly propylene glycol, poly ethylene glycol, gelatine, albumin, alginate, hydroxyl propyl methyl cellulose (HPMC), hydroxyl ethyl methyl cellulose (HEMC), bentonite, tragacanth, dextrin, sesame oil, almond oil, sucrose, acacia gum and xanthan gum and combinations thereof.

The carrier may, optionally, contain one or more plasticiser. Thus the carrier may also include a plasticiser. The plasticiser may, for example, be polyethylene glycol (PEG), polypropylene glycol, poly (lactic acid) or poly (glycolic acid) or a copolymer thereof, polycaprolactone, and low molecule weight oligomers of these polymers, or conventional plasticisers, such as, adipates, phosphates, phthalates, sabacates, azelates and citrates. The plasticiser may also be an alcohol such as ethanol or methanol.

The carrier may also include other known pharmaceutical excipients in order to improve the stability of the agent.

In one embodiment, one or more additional excipient or delivery enhancing agent may also be included e.g. surfactants and/or hydrogels, in order to further influence release rate.

Preferably the injectable scaffold material is capable of solidifying/self-assembling on/or after injection into a subject to form a scaffold. The scaffold is preferably porous. Preferably the pores are formed by the gaps which are left between particles used to form the scaffold. Preferably the scaffold has pore volume of at least about 50%. Preferably the pores have an average diameter of about 100 microns.

As the skilled man would appreciate, pore volume and pore size can be determined using microcomputer tomography (microCT) and scanning electron microscopy (SEM). For example, SEM can be carried out using a Phillips 535M SEM instrument.

The formation of porous scaffolds is described in WO2004/084968.

Preferably the agent release is controlled, that is, not all of the agent is released in one large dose. Preferably the scaffold produced permits the kinetics of agent release from the carrier to be controlled. The rate of release may be controlled by controlling the size and/or number of the pores in the scaffold and/or the rate of degradation of the scaffold. Other factors that can be controlled are the concentration of any suspending agent included in the carrier, the viscosity or physiochemical properties of the composition, and the choice of carrier.

The agent may be released by one or more of: diffusion of the agent through the pores; degradation of the scaffold leading to increased porosity and improved outflow of fluid carrying the agent; and physical release of agent from the particles. It is within the abilities of the skilled man to appreciate that the size and/or number of the pores in the scaffold and/or the rate of degradation of the scaffold can readily be selected by appropriate choice of starting material so as to achieve the desired rate of release.

Diffusion of the agent away from the scaffold occurs due to diffusion driven by a concentration gradient and the natural flow of body fluids through and away from the scaffold.

Preferably the scaffold has pores in the nanometer to millimeter range, preferably about 20 to about 50 microns. Preferably the scaffold has pores with an average size of 100 microns. Preferably the scaffold has a least about 30%, about 40%, about 50% or more pore volume.

The system of the invention may allow for agent release to be sustained for some time, preferably at least about 2 hours, at least about 4 hours, at least about 6 hours, at least about 10 hours, at least about 12 hours, at least about 24 hours, more preferably at least 48 hours, preferably at least a week, preferably more than one week, preferably more than 10 days.

Preferably the agent is released in an amount effective to have a desired local or systemic physiological or pharmacologically effect.

Preferably delivery of an agent means that the agent is released from the scaffold into the environment around the scaffold, for example surrounding tissues.

Preferably the composition of the invention allows a substantially zero or first order release rate of the agent from the scaffold once the scaffold has formed. A zero order release rate is a constant release of the agent over a defined time; such release is difficult to achieve using known delivery methods.

Preferably, the initial day 1 burst release is less than about 25-33% of total loading (ideally less than about 20% or more ideally less than about 10% or more ideally less than about 5%). This initial burst is preferably then followed by 1-2% release per day for about 14 days (which may equate to about 0.5-2 mcg/day). Preferably, release of drug continues for at least 14 days and preferably for at least 20 days, 30 days, 40 day or 50 days. In some embodiments, release continues for about 14 to 56 days. In some embodiments release continues for more than 56 days.

The release kinetics of the drug may be further modified by a number of means apparent to the skilled person. For example, adjustments to the PLGA copolymer ratio, end groups, molecular weight and/or particle size can all have an impact upon the release kinetics. The skilled person is able to determine by empirical studies appropriate combinations of these factors to provide the desired release profile

In alternative embodiments, drug release kinetics may be accelerated by adjusting the hydrophilicity of the polymer (e.g. by encapsulating the drug directly into the particles which are capable of interacting to form a scaffold and modifying the degradation profile by blending with PEG, or by modifying drug-loaded particles with PGA-PEG triblock co-polymers).

In other embodiments, release kinetics can be modified by the use of mixed molecular weight PLGA polymers, which can effectively increase either the initial or longer-term release and help to avoid any therapeutic lag phase (European Journal of Pharmaceutics and Biopharmaceutics Volume 50, Issue 2, September 2000, Pages 263-270).

In other embodiments other release modifiers may be used to adjust release kinetics. For example, adjustments to the viscosity of a carboxymethycellulose-containing liquid phase residing within the scaffold pores may be made.

By using a composition which solidifies to form a scaffold after administration, a scaffold can be formed which conforms to the shape of where it is placed, for example, the shape of a tissue cavity into which it is placed. This overcomes a problem with scaffolds fabricated prior to administration which must be fabricated to a specific shape ahead of administration, and cannot be inserted through a bottle-neck in a cavity and cannot expand to fill a cavity.

Preferably the composition is intended to be administered by injection into the body of a human or non-human animal. If the composition is injected then the need for invasive surgery to position the scaffold is removed.

Preferably the composition is sufficiently viscous to allow administration of the composition to a human or non-human animal, preferably by injection. Preferably the composition is intended to be administered at room temperature, and is preferably viscous at room temperature. The term room temperature is intended to refer to a temperature of from about 15° C. to about 25° C., such as from about 20° C. to about 25° C.

Alternatively, the composition may be heated to above room temperature, for example to body temperature (about 37° C.) or above, for administration. The composition is preferably flowable or viscous at this temperature in order to aid its administration to a human or non-human animal.

Preferably the composition has a viscosity which allows it to be administered, using normal pressure, from a syringe which has an orifice of about 4 mm or less. The size of the orifice will depend on the medical application, for example, for many bone applications a syringe with an orifice of between about 2 mm and about 4 mm will be used, however, for other applications smaller orifices may be preferred. Preferably “normal pressure” is that applied by a human administering the composition to a patient using one hand.

Preferably the composition is of sufficient viscosity such that when it is administered it does not immediately dissipate, as water would, but instead takes the form of the site where it is administered. Preferably some of the carrier and agent will dissipate from the scaffold over time.

In one embodiment, the composition is sufficiently viscous that when administered the injectable scaffold material remain substantially where it is injected, and do not immediately dissipate. Preferably, the scaffold forms before there has been any substantial dissipation of the injectable scaffold material. Preferably more than about 50%, 60% 70%, 80% or 90% by weight of the injectable scaffold material injected into a particular site will remain at the site and form a scaffold at that site.

In a preferred embodiment the injectable scaffold material is capable of spontaneously solidifying when injected into the body due to an increase in temperature post administration (e.g. increase in the temperature from room temperature to body temperature). This increase in temperature may cause the injectable scaffold material to interact to form a scaffold.

Preferably when a composition solidifies to form a scaffold it changes from a suspension or deformable viscous state to a solid state in which the scaffold formed is self-supporting and retains its shape. The solid scaffold formed may be brittle.

Solidification of the injectable scaffold material may be triggered by any appropriate means, for example, solidification may be triggered by a change in temperature, a change in pH, a change in mechanical force (compression), or the introduction of a cross-linking, setting or gelling agent or catalyst.

In other words, the particles may be particles, such as polymer particles, that can be solidified by a change in temperature, a change in pH, a change in mechanical force (compression), or the introduction of a cross-linking agent, setting agent or gelling agent or catalyst.

The injectable scaffold material may be cross linked by a variety of methods including, for example, physical entanglement of polymer chains, UV cross linking of acrylate polymers, Michael addition reaction of thiolate or acrylate polymers, thiolate polymers cross linked via vinyl sulphones, cross linking via succinimates of vinyl sulphones, cross linking via hydrazines, thermally induced gelation, enzymatic crosslinking (for example, the addition of thrombin to fibrinogen), cross linking via the addition of salts or ions (especially Ca²⁺ ions), cross linking via isocyanates (for example, hexamethylene diisocyanate).

The injectable scaffold material comprises discrete particles, which are capable of interacting to form a scaffold. The interaction may cause the particles to cross link, wherein the particles become physically connected and are held together. Cross linking may be achieved by covalent, non-covalent, electrostatic, ionic, adhesive, cohesive or entanglement interactions between the particles or components of the particles.

Accordingly, it is preferred that the discrete particles are capable of cross linking, such that the particles become physically connected and are held together. The particles may suitably be polymer particles that are capable of cross linking, such that the particles become physically connected and are held together.

The preferred characteristic for the particles, to ensure a scaffold can be formed, is the glass transition temperature (Tg). By selecting particles that have a Tg above room temperature, at room temperature the particles are below their Tg and behave as discrete particles, but when exposed to a higher temperature (e.g. in the body) the particles soften and interact/stick to their neighbours. Preferably particles are used that have a Tg from about 25° C. to 50° C., such as from about 27° C. to 50° C., e.g. from about 30° C. to 45° C., such as from 35° C. to 40° C., for example from about 37° C. to 40° C.

As the skilled man would appreciate, glass transition temperatures can be measured by differential scanning calorimetry (DSC) or rheology testing. In particular, glass transition temperature may be determined with DSC at a scan rate of 10° C./min in the first heating scan, wherein the glass transition is considered the mid-point of the change in enthalpy. A suitable instrument is a Perkin Elmer (Bucks, United Kingdom) DSC-7.

In other words, the formation of the scaffold is caused by exposing the particles to a change in temperature, from a temperature that is below their Tg to a higher temperature. The higher temperature does not necessarily have to be equal to or above their Tg; any increase in temperature that is towards their Tg can trigger the required interaction between the particles. Preferably, the formation of the scaffold is caused by exposing the particles to a change in temperature, from a temperature that is below their Tg to a higher temperature, wherein the higher temperature is not more than 5° C. below their Tg, such as not more than 3° C. below their Tg or not more than 2° C. below their Tg or not more than 1° C. below their Tg.

Essentially, if polymer particles are raised close to or above their onset temperature on injection into the body, the polymer particles will cross-link to one or more other polymer particles to form a scaffold. By cross-link it is meant that adjacent polymer particles become joined together. For example, the particles may cross-link due to entanglement of the polymer chains at the surface of one particle with polymer chains at the surface of another particle. There may be adhesion, cohesion or fusion between adjacent particles.

When the particles come together and cross-link, pores are formed in the resultant scaffold, as a consequence of the inevitable spaces between adjacent particles.

In an embodiment the system comprise discrete particles which are capable of interacting to form a scaffold which have a Tg between about 35° C. and about 40° C., as well as other discrete particles that have a Tg about 40° C. The agent for delivery may be incorporated into just one of the particle types or both. Preferably the agent for delivery is incorporated in at least the discrete particles that have a Tg above 40° C.

The particles may be at least partially dispersible in the carrier. Preferably the particles are not soluble in the carrier at a temperature of 37° C. or less.

The carrier may interact with the particles. The carrier may interact with the particles to prevent or slow the formation of a scaffold and to allow the particles to be administered to a human or non-human animal before a scaffold forms. The carrier may prevent interaction between the particles due to separation of the particles by suspension in the carrier. It may be that the carrier completely prevents the formation of the scaffold prior to administration, or it may simply slow the formation, e.g. permitting the scaffold formation to begin but not complete formation prior to administration. In one embodiment the composition comprises sufficient carrier to prevent the formation of a scaffold even when the composition is at a temperature which, in the absence of the carrier, would cause the particles to form a scaffold. In one embodiment, the composition comprises sufficient carrier to slow the formation of a scaffold such that when the composition is at a temperature which, in the absence of the carrier, would cause the polymer particles to readily form a scaffold, a scaffold does not readily form, e.g. does not form over a timescale such as one hour to five hours.

The carrier may interact with the particles and cause the surface of the particles to swell, whilst remaining as discrete particles, thus allowing administration by injection. However, once the composition has been administered and the carrier begins to dissipate the particles may begin to de-swell. De-swelling may assist the joining together of particles.

Interaction of the polymer particles with the carrier may cause the glass transition temperature of the particles to change. For example, the interaction may cause the glass transition temperature to be lowered.

The carrier may act as a lubricant to allow the particles to be administered to a human or non-human animal, preferably by injection. Preferably the carrier provides lubrication when the composition is dispensed from a syringe. The carrier may help to reduce or prevent shear damage to particles dispensed from a syringe.

The discrete particles may be of one or more polymer, preferably one or more synthetic polymer. The particles may comprise one or more polymer selected from the group comprising poly (α-hydroxyacids) including poly (D,L-lactide-co-glycolide)(PLGA), poly D,L-lactic acid (PDLLA), polyethyleneimine (PEI), polylactic or polyglcolic acids, poly-lactide poly-glycolide copolymers, and poly-lactide poly-glycolide polyethylene glycol copolymers, polyethylene glycol (PEG), polyesters, poly (ε-caprolactone), poly (3-hydroxy-butyrate), poly (s-caproic acid), poly (p-dioxanone), poly (propylene fumarate), poly (ortho esters), polyol/diketene acetals addition polymers, polyanhydrides, poly (sebacic anhydride) (PSA), poly (carboxybiscarboxyphenoxyphosphazene) (PCPP), poly [bis (p-carboxyphenoxy) methane] (PCPM), copolymers of SA, CPP and CPM (as described in Tamat and Langer in Journal of Biomaterials Science Polymer Edition, 3, 315-353. 1992 and by Domb in Chapter 8 of The Handbook of Biodegradable Polymers, Editors Domb A J and Wiseman R M, Harwood Academic Publishers), poly (amino acids), poly (pseudo amino acids), polyphosphazenes, derivatives of poly [(dichloro) phosphazene], poly [(organo) phosphazenes], polyphosphates, polyethylene glycol polypropylene block co-polymers for example that sold under the trade mark Pluronics™, natural or synthetic polymers such as silk, elastin, chitin, chitosan, fibrin, fibrinogen, polysaccharides (including pectins), alginates, collagen, peptides, polypeptides or proteins, copolymers prepared from the monomers of any of these polymers, random blends of these polymers, any suitable polymer and mixtures or combinations thereof.

Preferably the particles comprise polymer selected from the group comprising poly(α-hydroxyacids) such as poly lactic acid (PLA), polyglycolic acid (PGA), poly(D,L-lactide-co-glycolide)(PLGA), poly D, L-lactic acid (PDLLA), poly-lactide poly-glycolide copolymers, and combinations thereof.

More preferably the particles comprise polymer which is a blend of a poly(α-hydroxyacid) with poly(ethylene glycol) (PEG), such as a blend of a polymer or copolymer based on glycolic acid and/or lactic acid with PEG.

The particles may be biocompatible and/or biodegradable. By controlling the polymers used in the particles the rate of scaffold degradation may be controlled.

The injectable scaffold material may comprise one or more type of polymer particle made from one or more type of polymer.

Where more than one type of particle is used each particle may have a different solidifying or setting property. For example, the particles may be made from similar polymers but may have different gelling pHs or different melting temperatures or glass transition points.

Preferably, in order for the polymer particles to form a scaffold the temperature around the particles, for example in the human or non-human animal where the composition is administered, is approximately equal to, or greater than, the glass transition temperature of the polymer particles. Preferably, at such temperatures the polymer particles will cross-link to one or more other polymer particles to form a scaffold or matrix. By cross-link it is meant that adjacent polymer particles become joined together. For example, the particles may cross-link due to entanglement of the polymer chains at the surface of one particle with polymer chains at the surface of another particle. There may be adhesion, cohesion or fusion between adjacent particles.

Preferably the injectable scaffold material comprises particles which are formed of a polymer or a polymer blend that has a glass transition temperature (Tg) either close to or just above body temperature (such as from about 30° C. to 45° C., e.g. from about 35° C. to 40° C., for example from about 37° C. to 40° C.). Accordingly, at room temperature the particles are below their Tg and behave as discrete particles, but in the body the particles soften and interact/stick to their neighbours. Preferably scaffold formation begins within 15 minutes of the raise in temperature from room to body temperature.

The particles may be formed from a polymer which has a Tg from about 35° C. to 40° C., for example from about 37° C. to 40° C., wherein the polymer is a poly(α-hydroxyacid) (such as PLA, PGA, PLGA, or PDLLA or a combination thereof), or a blend thereof with poly(ethylene glycol) (PEG). Preferably at body temperature these particles will interact to from a scaffold. The injectable scaffold material may comprise only poly(α-hydroxyacid)/PEG particles or other particle types may be included.

The particles may be formed from a blend of poly(D,L-lactide-co-glycolide)(PLGA) and poly(ethylene glycol) (PEG) which has a Tg at or above body temperature. Preferably at body temperature these particles will interact to from a scaffold, and during this process PEG may be lost from the surface of the particles which will have the effect of raising the Tg and hardening the scaffold structure. The injectable scaffold material may comprise only PLGA/PEG particles or other particle types may be included.

The composition may comprise a mixture of temperature sensitive particles and non-temperature sensitive particles. Preferably non-temperature sensitive particles are particles with a glass transition temperature which is above the temperature at which the composition is intended to be used. Preferably, in a composition comprising a mixture of temperature sensitive particles and non-temperature sensitive particles the ratio of temperature sensitive to non-temperature sensitive particles is about 3:1, or lower, for example, 4:3. The temperature sensitive particles are preferably capable of crosslinking to each other when the temperature of the composition is raised to or above the glass transition a temperature of these particles. By controlling the ratio of temperature sensitive particles to non-temperature sensitive particles it may be possible to manipulate the porosity of the resulting scaffold.

In one embodiment, ceramic particles may additionally be present in the composition. This will typically be a temperature insensitive particle type. Alternatively or additionally, polymer particles in the composition may themselves contain a ceramic component. This will typically be a temperature insensitive particle type.

The inclusion of ceramic material either as separate particles or within the polymer particles may enhance osteoconductivity and/or add osteoinductivity.

The particles may be solid, that is with a solid outer surface, or they may be porous. The particles may be irregular or substantially spherical in shape.

The polymer particles may have a size in their longest dimension, or their diameter if they are substantially spherical, of less than about 3000 μm and preferably more than about 1 μm. More preferably the particles have a size in their longest dimension, or their diameter, of less than about 1000 μm. Preferably the particles have a size in their longest dimension, or their diameter, of between about 50 μm and about 500 μm, more preferably between about 100 μm and about 500 μm. Preferably polymer particles of the desired size are unable to pass through a sieve or filter with a pore size of about 50 μm, but will pass through a sieve or filter with a pore size of about 500 μm. More preferably polymer particles of the desired size are unable to pass through a sieve or filter with a pore size of about 200 μm, but will pass through a sieve or filter with a pore size of about 500 μm.

Formation of the scaffold from the composition, once administered to a human or non-human animal, preferably takes from about 20 seconds to about 24 hours, preferably between about 1 minute and about 5 hours, preferably between about 1 minute and about 1 hour, preferably less than about 30 minutes, preferably less than about 20 minutes. Preferably the solidification occurs in between about 1 minute and about 20 minutes from administration.

Preferably the composition comprises from about 20% to about 80% injectable scaffold material and from about 20% to about 80% carrier; from about 30% to about 70% injectable scaffold material and from about 30% to about 70% carrier; e.g. the composition may comprise from about 40% to about 60% injectable scaffold material and from about 40% to about 60% carrier; the composition may comprise about 50% injectable scaffold material and about 50% carrier. The aforementioned percentages all refer to percentage by weight.

Preferably the composition can be used to form a scaffold that can resist a compressive load in excess of 2 MPa (thus is suitable for bone applications).

Preferably the scaffold forms without the generation of heat or loss of an organic solvent.

The composition of the injectable agent delivery system may be for use in a method of treatment of the human or animal body by surgery or therapy or in a diagnostic method practised on the human or animal body. The composition of the injectable agent delivery system may be for pharmaceutical use or may be for use in cosmetic surgery.

The invention also provides, in a further aspect, a method of forming a scaffold comprising:

(1) providing a product as described herein; and
(2) allowing the discrete particles to solidify or self-assemble to form a scaffold having pores.

The method may be practised on tissue in vivo or in vitro.

Solidification of the discrete particles into a scaffold may, for example, be triggered by a change in temperature, a change in pH, a change in mechanical force, or the introduction of a cross-linking agent, setting agent, gelling agent or catalyst. In one embodiment, solidification of the scaffold material comprising discrete particles into a scaffold is caused by exposing the particles to a change in temperature, from a temperature that is below their Tg to a higher temperature.

In a further aspect, the invention provides a method of delivering an agent to a subject comprising providing an injectable scaffold material, wherein the agent is located within discrete particles with in the scaffold material; administering the scaffold material to a subject; allowing the scaffold material to solidify/self-assemble in the subject to form a scaffold; and allowing the agent contained within the scaffold material to be released into the subject at the site of administration.

The method may be practised on tissue in vivo or in vitro.

The agent (encapsulated within discrete particles) may optionally be added to the injectable scaffold material immediately prior to administration to the subject.

In one embodiment, in step d) the agent release is sustained over a period at least 12 hours.

Solidification of the scaffold material into a scaffold may, for example, be triggered by a change in temperature, a change in pH, a change in mechanical force, or the introduction of a cross-linking agent, setting agent, gelling agent or catalyst. In one embodiment, solidification of the scaffold material comprising discrete particles into a scaffold is caused by exposing the particles to a change in temperature, from a temperature that is below their Tg to a higher temperature.

According to a yet further aspect, the invention provides a scaffold produced by any method of the invention.

According to another aspect, the invention provides an injectable scaffold material as described with reference to the first aspect of the invention.

The scaffold formed by any method and/or composition of the invention may be used to treat damaged tissue. In particular, the scaffold may be used to encourage or allow cells to re-grow in a damaged tissue. The invention may therefore be used in the treatment of tissue damage, including in the regeneration or reconstruction of damaged tissue.

The composition of the invention may be used to produce scaffolds for use in the treatment of a disease or medical condition, such as, but not limited to, Alzheimer's disease, Parkinson's disease, osteoarthritis, burns, spinal disk atrophy, cancers, hepatic atrophy and other liver disorders, bone cavity filling, regeneration or repair of bone fractures, diabetes mellitus, ureter or bladder reconstruction, prolapse of the bladder or the uterus, IVF treatment, muscle wasting disorders, atrophy of the kidney, organ reconstruction and cosmetic surgery.

According to a yet further aspect, the invention provides a method of treating a subject, such as a mammalian organism, to obtain a desired local physiological or pharmacological effect comprising administering an injectable agent delivery system according to the invention to a site in the subject (e.g. the organism) in need of such treatment. Preferably the method allows the agent to be delivered from the scaffold to the area surrounding the site of scaffold formation.

According to a further aspect, the invention provides the use of a composition according to the invention as an injectable scaffold material in tissue regeneration and/or in the treatment of tissue damage.

The product of the invention may be used for the treatment or prevention of a condition selected from: neurodegeneration disorders (e.g. post stroke, Huntington's, Alzheimer's disease, Parkinson's disease), bone-related disorders (including osteoarthritis, spinal disk atrophy, bone cavities requiring filling, bone fractures requiring regeneration or repair), burns, cancers, liver disorders (including hepatic atrophy), kidney disorders (including atrophy of the kidney), disorders of the bladder, ureter or urethra (including damaged ureter or damaged bladder requiring reconstruction, prolapse of the bladder or the uterus), diabetes mellitus, infertility requiring IVF treatment, muscle wasting disorders (including muscular dystrophy), cardiac disorders (e.g. damaged cardiac tissue post myocardial infarction, congestive heart disease), eye disorders (e.g. damaged or diseased cornea), damaged vasculature requiring regeneration or repair, ulcers, and damaged tissue requiring regeneration or reconstruction (including damaged organ requiring regeneration or reconstruction, and damaged nerves requiring regeneration or reconstruction).

According to another aspect, the invention provides a kit for use in delivering an agent to a target comprising a composition according to the invention and instructions to use the composition.

The kit may include a syringe for use in injecting the composition. The composition may be provided preloaded in the syringe, ready for use. Preferably the kit can be stored either refrigerated or at room temperature.

According to further aspect, the invention provides a method of producing a composition for an injectable agent delivery system, said method comprising: adding the agent for delivery to a melt blended thermosetting polymer; adding the agent for delivery to a melt blended thermosetting polymer; allowing the melt blended thermosetting polymer to cool and set; and creating particles from the set melt blended thermosetting polymer, for example by milling, die-cutting of extruded polymer, or spheronisation.

In yet a further aspect the invention provides a method of producing a composition for an injectable agent delivery system, said method comprising: encapsulating the agent for delivery into particles of a non-thermosetting polymer; and combining the resulting particles with thermosetting polymer particles.

In a further aspect, the invention provides a method of producing a composition for an injectable agent delivery system, said method comprising: encapsulating the agent for delivery into particles of a non-thermosetting polymer; combining resulting particles with a thermosetting polymer; melting the thermosetting polymer to embed the particles from the first step therein and allowing the combination to set; and creating particles from the set combination. Particles may be created, for example, by milling, die-cutting of extruded polymer or spheronisation.

The skilled man will appreciate that the preferred features of the first aspect, or any aspect, of the invention can be applied to all aspects of the invention.

Embodiments of the invention will now be described, by way of example only, with reference to the following examples. The examples refer to the Figures in which:

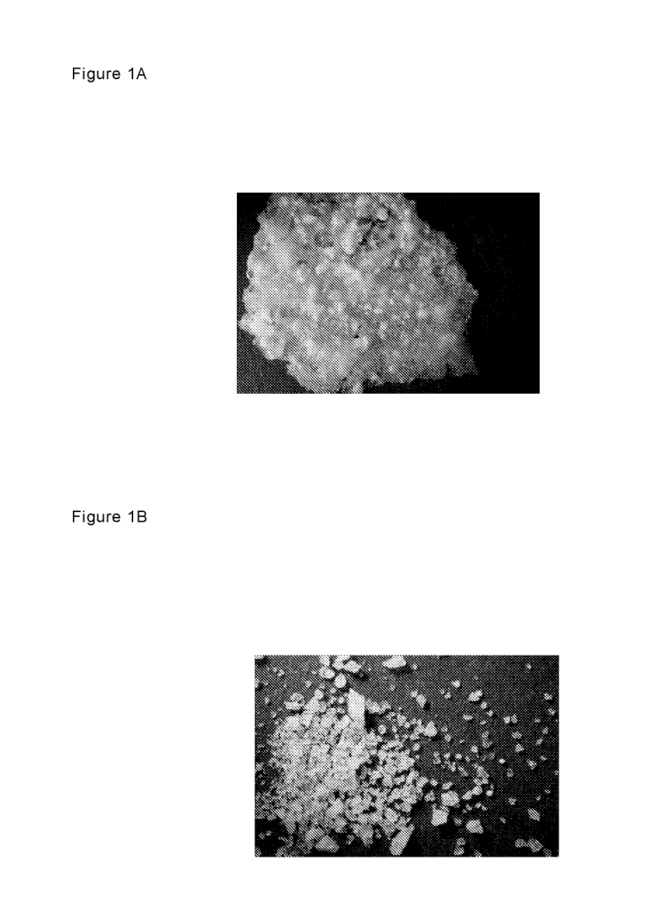

FIG. 1A shows placebo microspheres embedded within melted PLGA/PEG.

FIG. 1B shows the particles created from the melt shown in FIG. 1.

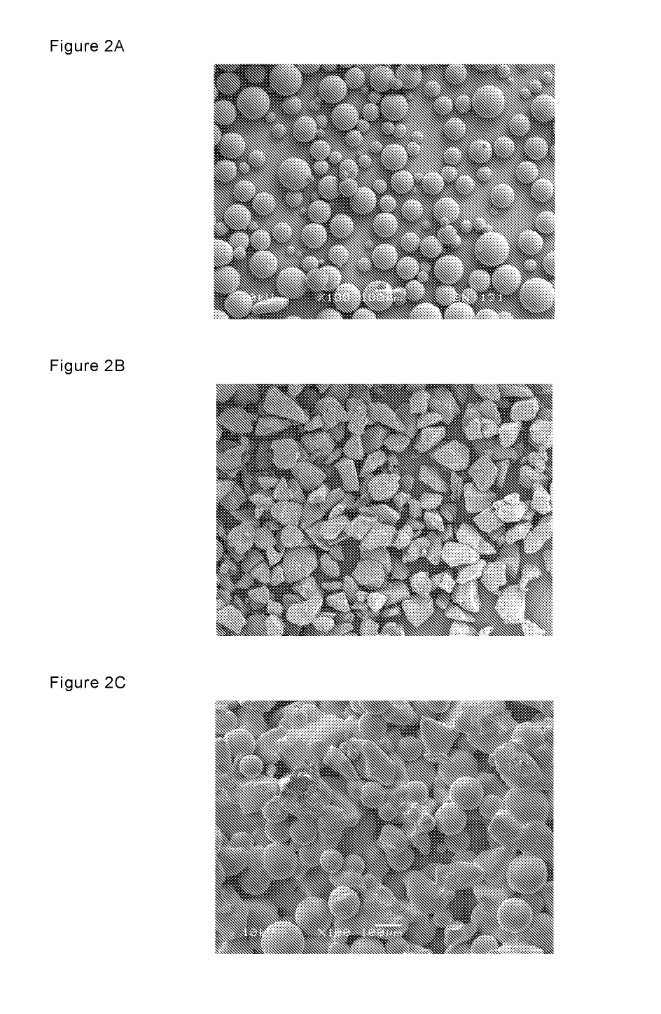

FIG. 2A shows spherical particles made by an emulsion method of PLGA 50:50 (Mwt 56). The particles are loaded with simvastatin. The particles have a glass transition temperature above 40° C.

FIG. 2B shows rough milled microparticles manufactured from PLGA 85:15 (Mwt 50 KDa) blended with 6.5% w/w polyethylene glycol 400 Da below their glass transition temperature.

FIG. 2C shows a scaffold of the particles on FIGS. 2A and 2B after they have been mixed and heated to above the glass transition temperature of the particles in FIG. 2B and below the glass transition temperature of the particles of FIG. 2A.

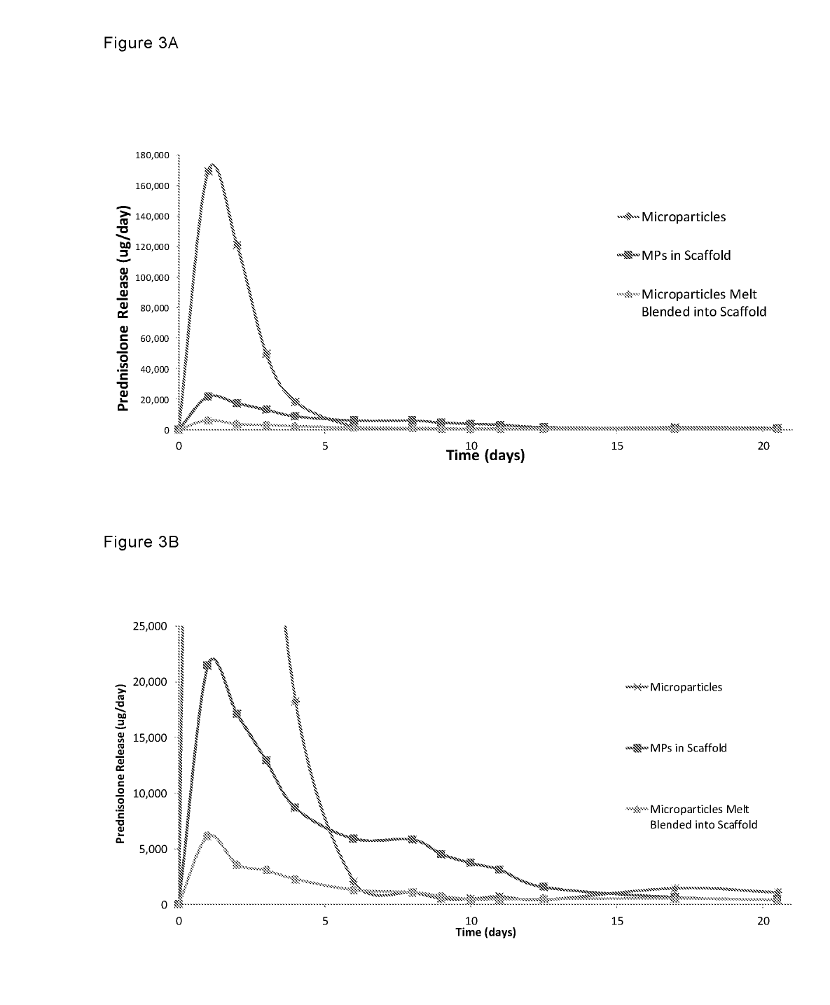

FIG. 3A shows the results of prednisolone release experiments from microparticles, microparticles in scaffold and microparticles melt blended into scaffold, with data normalized to a minimum daily dose of 400 μg (the minimum therapeutic dose).

FIG. 3B shows the same data as FIG. 3A, but enlarged for clarity.

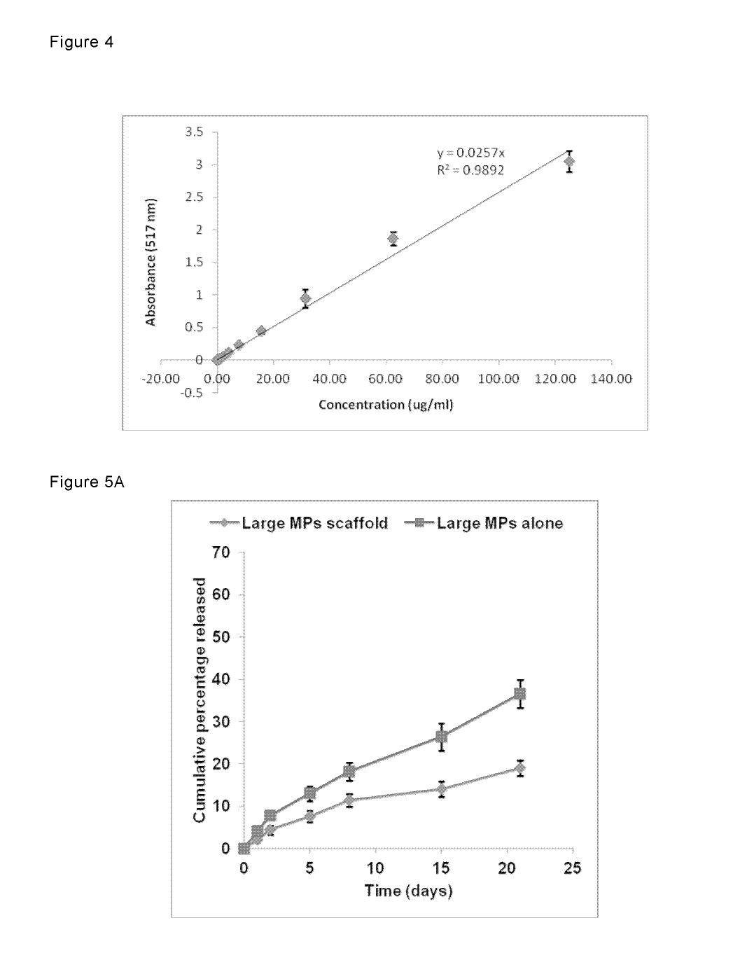

FIG. 4 shows a calibration curve for the absorbance at 517 nm for Oil Red-O.

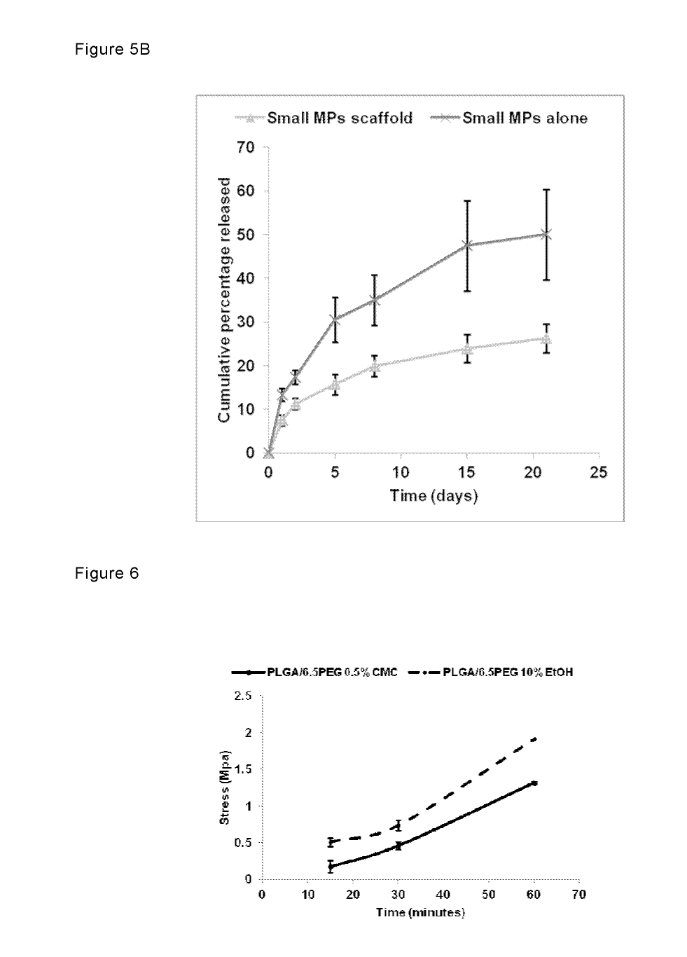

FIG. 5A shows the results of experiments to shown Oil Red-O release from large (50-10 μm) microparticles alone or in a scaffold.

FIG. 5B shows the results of experiments to shown Oil Red-O release from small (20-3 μm) microparticles alone or in a scaffold.

FIG. 6 shows a comparison of compressive strength of PLGA/6.5% scaffolds having 0.5% CMC or 10% ethanol as a carrier.

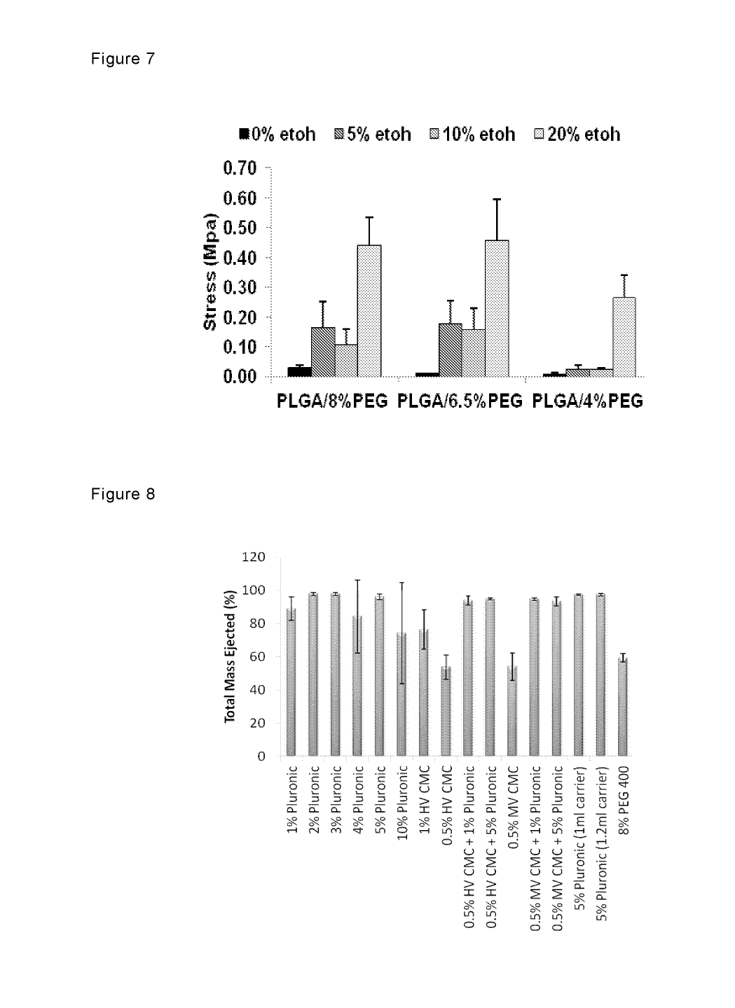

FIG. 7 shows a comparison of compressive strength of PLGA/PEG scaffolds made with a variety of carriers after 15 minutes of sintering at 37° C.

FIG. 8 shows the injection yield of injectable scaffolds formulated with different carriers.

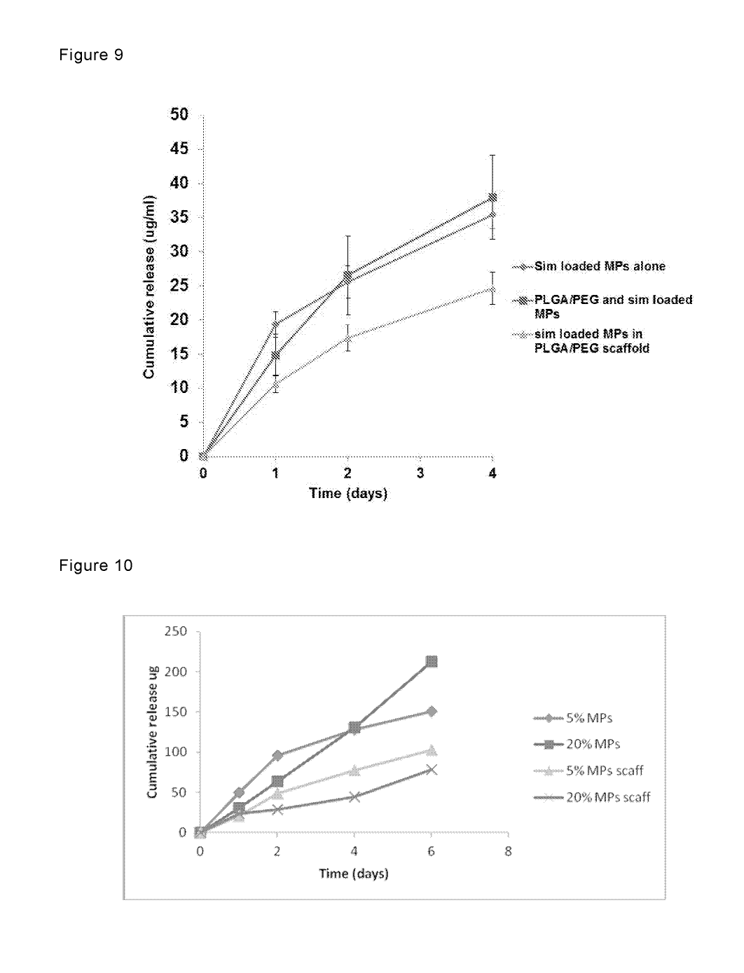

FIG. 9 shows release of simvastatin from microparticles alone, PLGA/PEG combined with simvastatin loaded microparticles and simvastatin loaded microparticles in a PLGA/PEG scaffold.

FIG. 10 shows release of simvastatin from microparticles loaded with 5% and 20% simvastatin, either alone or in PLGA/PEG scaffold.

### EXAMPLES

### Example 1

### Melt Entrapment of Microparticles within Thermosetting PLGA/PEG Particles

1. Preparation of PLGA/PEG sheet:
1.1 2.5 g PLGA/PEG particles (of a size of <100 microns and containing either 6.5% w/w or 8% w/w 400 Da PEG) were weighed out into a weigh boat.
1.2 In a separate weigh boat, 0.278 g (i.e. 10% w/w of total combined mass) PLGA ‘placebo’ particles (20-50 micron diameter spherical particles) was weighed, before adding to the PLGA/PEG particles and mixing thoroughly with a spatula
1.3 The contents of the weigh boat were poured onto a PTFE sheet and flattened with a spatula to an approximately 2 mm depth layer of continuous particles (FIG. 1)
1.4 The PTFE sheet was placed into an oven at 50° C. and left for approximately 6 hours. A thin sheet of clear PLGA/PEG will be formed.
   2. Preparation of Thermosetting PLGA/PEG blend Particles containing Placebo Particles
2.1 Using scissors cut the polymer sheet into pieces of less than 2×2 cm.
2.2 Cool a Krups 75 grinder by filling the metal section only of the grinder with liquid nitrogen. Once all liquid nitrogen is vaporised, place the shards into the grinder.
2.3 Mill the particles in short 5 second bursts. The particles must be sieved to produce all particles below 300 μm. Prolonged milling can generate heat which may cause the polymer to melt.
2.4 Once all particles are below 300 μm in size, they are placed onto the PTFE sheet once again to an approximately 2 mm depth layer of continuous particles.
2.5 The particles are placed into an oven at 50° C. once again for 6 hours. Steps 2.1 to 2.3 are repeated but the particles are milled into the size fractions of 100-200, 200-300 and 300-400 μm. The resultant particles (FIG. 2) are then stored at less than 4° C. until required.

This method provides for placebo spherical particles blended with <100 micron fraction 6.5% as a powder blend, before heating to either 45° C. for 21 hrs, or 50° C. for 6 hrs. The 8% PEG formulation was sufficiently melted for blending after 5 hrs at 50° C.

The above method provides for melt blending spherical, non-thermosetting PLGA particles (to which drugs could be easily added using standard emulsion methods) into PLGA/PEG. The blending provides a further barrier (i.e. more surrounding polymer) to drug release, and the PLGA particles protect the drug from the high temperatures they would otherwise face if added directly into a PLGA/PEG melt blend (at around 100 degC+).

### Example 2

### Reducing Cmax: Release Study

### Comparison of Drug-Loaded PLGA Microparticle Formulations:

This example shows a comparison of microparticles (MPs) vs MPs entrapped in a PLGA/PEG particle scaffold vs particles made by melt blending MPs into PLGA/PEG.

Model active was incorporated into the scaffold with the aim of better controlling drug release by reducing the initial ‘burst’ effect inherent in polymeric controlled release systems. Burst can be significantly reduced by formulation strategy, as shown using prednisolone loaded polymer microspheres incorporated in scaffolds.

### Methods:

The following solid dispersion emulsion method was used to make PLGA particles incorporating Prednisolone:

1 litre batches of PVA 0.3% (w/v) solution in distilled water were fully dissolved overnight on a magnetic stirrer set to ca. 200 rpm. Before use, the PVA solution was passed through a 0.2 micron filter unit under vacuum to remove any particulate matter.

For each batch, 1±0.01 g of polymer was weighed into glass vials. 6.67 ml of DCM was added to the vials to give a 15% (w/v) polymer solution. The vials were left on an orbital shaker for at least 30 minutes to enable the polymer to dissolve.

Predisolone (200±2 mg) was transferred to a vial containing polymer/DCM. All of the vials were sonicated on ice using a probe sonicator (Soniprep 150 from MSE Ltd. Crawley, Sussex, UK) for 1 minute with a frequency of 15 Kilohertz without pulsing to disperse the insoluble steroids within the organic phase.

200 ml of 0.3% PVA solution was added to a beaker and positioned underneath the Silverson homogeniser (model L5M) rotor on a screw jack. The rotor was lowered into the solution then the position of the rotor head adjusted to 2 cm from the base of the beaker.

The rotor, fixed with a Silverson Square Hole High Shear Screen™, was set to spin at 2,000 rpm then the contents of the vial was poured into the PVA bath. After two minutes, the beaker was removed, and a glass magnetic stirrer (50 mm) added to the beaker, which was then placed onto a multi-way magnetic stirrer set at 300 rpm for four hours.

Once DCM evaporation was complete (four hours), the beaker was removed from the magnetic stirrer, and the resultant particles then washed over a 0.2 micron filter using 2 litres of distilled water. The particles were passed through a 100 micron strainer into a 50 ml centrifuge tube, freeze dried for >96 hours and stored vacuum packed in the freezer.

20% w/w of prednisolone drug loaded particles (DLP) were mixed with PLGA/PEG 100-200 μm particles and warmed above body temperature to set and entrap the DLP. Further scaffolds were also made after first melt encapsulating the DLP within PLGA/PEG 100-200 μm particles.

Predisolone-loaded particles (control) and DLP scaffold samples were placed in graduated 22 ml glass vials (Supelco) in 20 ml of 0.5% v/v Tween 20 in 100 mM phosphate buffered saline and stored in a 37° C. incubator with magnetic stirring at 130 rpm. At each time point in the release study, the tubes were removed from the stirrer then 16 ml of supernatant was taken, and replaced with an equal volume of fresh 0.5% v/v Tween 20 in 100 mM phosphate buffered saline.

Samples were frozen after collection then thawed and vortexed before 250 μl of each sample was mixed with 75 μl HPLC grade methanol. This was then vortexed for 60 secs, and then centrifuged at 14,000 g to create a further supernatant, 60 μl of which was transferred to HPLC tubes for analysis.

HPLC analysis was conducted using a Hypersil C18 column (100 mm, i.d 5 mm, particle size 5 μm; ThermoFisher) and Beckman HPLC. All samples were run using a sample injection volume of 5 μm, and column temperature of 40° C. An isocratic mobile phase of 60% methanol and 40% dH₂O was used at a flow rate of 1 ml/min, with detection at a wavelength of 254 nm Concentration curves were prepared using serial dilutions of test samples and measurement of the area under the curve (AUC) for each detected peak.

Results are shown in FIG. 3 and are tabulated below.

| | | Relative % Cmax | |
|---|---|---|---|
| Formulation | Cmax (μg) | % of A | % of B |
| Microparticles (A) | 168,847 | — | — |
| MPs in TSP (B) | 21,489 | 12.7% | — |
| MPs Melt Blended into TSP (C) | 6,129 | 3.6% | 28.5% |

(A) = DLP only,
(B) = DLP entrapped in PLGA/PEG scaffold,
(C) = DLP melt entrapped in PLGA/PEG as per method in Example 1 and then milled into particles and scaffold made

### Example 3

### Release of Oil Red-O as a Model Lipophilic Drug (Mwt 408, log P 6.85)

### Aim:

To determine the release profiles from oil Red-O (ORO) loaded microparticles (MPs) over time and to compare the release rates of small (20-30 micron) MPs with larger (50-100 micron) MPs.

Also to determine whether incorporation into a PLGA/PEG scaffold will reduce the burst release and provide a more sustained release over time.

### Method:

The PLGA/PEG blend used was made by incorporating 8% PEG400 into PLGA 85:15 (LP671). PLGA microparticles were prepared using a DCM emulsion method and a 1% loading of ORO in microparticles was used. Therefore each 50 mg particles contains a maximum of 500 mg drug.

### Scaffold Manufacture:

300 mg of PLGA/8% PEG 100-200 micron size fraction particles were weighed into a weigh boat and 100 mg of ORO MPs were added and manually mixed.

320 ml of liquid carrier (1% pluronics F127, 0.5% CMC, 20% ethanol in phosphate buffered saline) was added to the particles and thoroughly mixed (0.8:1 carrier to particle ratio). The paste was evenly split between two PTFE disc moulds so each scaffold would have approximately 50 mg of ORO MPs and 150 mg of PLGA/8% PEG particles. This was repeated for blank large MPs, ORO loaded small MPs, and blank small MPs. The particles in the PTFE moulds were sintered by placing in an oven for 1 hour at 37° C.

### Release Assay:

After sintering, the scaffolds were removed from the mould and placed into 10 ml of PBS containing 0.5% tween-20 (20 ml tube). Separate tubes containing 10 ml of PBS/tween were prepared with 50 mg of MPs alone (20 ml tube). The tubes were placed on a 3D rocker (Gyrotwister) set at 5 rpm. At specific time points, the tubes were removed from the rocker, the MPs alone were centrifuged at 3000 rpm for 5 minutes (Mistral). 9 ml of PBS-tween was collected from each tube and stored frozen. 9 ml of fresh PBS-tween was added back into each tube and re-incubated on the gyrotwister.

### Quantification of ORO in Release Supernatants:

The samples were thawed, 150 ml plated into a 96 flat well transparent plate and read at 517 nm on the Tecan plate reader. A standard curve was prepared by dissolving ORO at 1 mg/ml in methanol and diluting down in PBS-tween (NB diluting the samples in methanol did not improve their solubility but reduced the absorbance down to low levels. This was therefore not pursued further). All samples were tested on the same day. The release assay was stopped on day 21. The remaining scaffolds and MPs were treated with 5 ml methanol overnight to extract any remaining ORO.

Results are shown in FIGS. 4 and 5. Small DLPs release more quickly than larger DLPs (as expected due to the increased surface area for diffusion & degradation). In both systems, physical entrapment of the DLPs within PLGA/PEG scaffolds slows the release over the period of study i.e. both the initial burst (24-48 hrs where surface drug is released) and longer degradation controlled release. Both DLPs show approximately 50% suppression of release at the end of the study for the formulations tested.

### Example 4

### Changing Formulation of the Liquid Carrier

### METHOD A. Incorporation of Liquid Plasticiser

A liquid plasticiser was included into the carrier to create wider range of setting kinetics and physical characteristics of PLGA/PEG scaffolds.

The addition of a liquid plasticiser within the carrier phase of the material will:
improve the ambient temperature stability by allowing lower PEG content in the blended scaffold particles
allow faster setting of the scaffold enabling larger volumes of secondary particles (e.g. ceramics) to be added and avoiding migration of particles from delivery site

Scaffolds with further improved mechanical strength profiles are also made possible.

### Methods:

Initial candidates N-methyl-2-pyrrolidone (NMP) in water and triethyl citrate (TEC) in ethanol were mixed in various concentrations with PLGA 85:15.

### Results:

NMP was only effective at high concentrations whilst TEC was most effective at low concentrations. The ethanol diluent alone was therefore investigated further.

### The Use of Ethanol as a Liquid Plasticiser in the Carrier Phase of PLGA/PEG Scaffolds

### Methods:

Scaffolds were fabricated for compressive strength testing with PLGA/8% PEG MPs and either 0.5% CMC (std) or 50% ethanol as the carrier. Scaffolds were sintered for 5, 15 and 30 minutes at 37° C. prior to compressive strength testing. A lower ethanol (10%) and a lower PEG (6.5%) content were also tested.

### Results:

At each timepoint, scaffolds made with 50% ethanol were 4-5 times stronger than those made with the standard carrier. The lower concentration of ethanol (10%) also improved the compressive strength (FIG. 6). The use of ethanol provides greater scope over the formulation and attainable release profiles, allowing the use of lower PEG content in the blended scaffold particles. This facilitates slower degradation and release (since hydrophilic PEG draws water into PLGA and accelerates degradation rate). Faster setting of the scaffold using EtOH means larger volumes of secondary particles (i.e. DLPs) may be added if required.

### Example 5

### Combining Injectability with Compressive Strength of PLGA/PEG Scaffolds

### Method A:

PLGA/PEG MPs were mixed in opposing syringes with carriers comprised of 1% (w/v) Pluronics F127/0.5% (w/v) CMC (hv) with ethanol at 5, 10 and 20% (v/v). The paste was injected through a 19 ga needle and used to manufacture scaffolds for compressive testing.

### Results:

All formulations with ethanol had increased compressive strength (FIG. 7).

### Method B:

Injection yield and repeatability of injection through fine bore needles (19 ga) has been tested by reformulating the liquid carrier as shown in FIG. 8.

### Results:

Increased yield from 53% yield to 98% with minimal effect on scaffold strength was obtained (compared with the initial formulation containing 0.5% CMC (hv)). Best results were obtained by the use of high viscosity CMC/Pluronics as a suspending agent.

### Example 6

### Simvastatin Release Study

### Materials:

PLGA 5050 microparticles loaded with 1% w/w simvastatin, average size 85.53±25.52 micron and blank controls.

The drug-loaded particles were prepared as follows: A 1 litre batch of PVA was made up to a 0.3%_{(w/v)} solution in distilled water and was allowed to dissolve overnight on a 15-way magnetic stirrer set to 300 rpm. Before use the PVA solution was passed through a 0.2 micron filter unit under vacuum to remove any remaining particulate matter.

1±0.01 g of PLGA polymer was weighed into each of four glass vials. In addition, one vial had 50 mg of simvastatin added, one vial had 100 mg simvastatin added and one vial had 200 mg simvastatin added. This would equate to approximately 5,10 and 20%_{(w/w)} drug loading. The fourth vial had no simvastatin added. 5 ml of DCM was added to each of the vials (20%_{(w/v)} polymer solution). The vials were left on an orbital shaker set at a low rpm for at least 30 minutes for the polymer and drug to dissolve into a single phase.

200 ml of 0.3%_{(w/v)} PVA solution was added to four 250 ml beakers. One of these beakers was positioned underneath the Silverson rotor on a screw jack. The rotor was lowered to just submerge in the liquid then the position of the rotor head was adjusted to 2 cm from the base by using the screw jack.

The rotor fixed with the standard emulsifier screen was set to spin at 2,000 rpm then the contents of the PLGA only vial was poured into the PVA bath and the timer set for two minutes. Once the emulsification was complete, a glass magnetic stirrer (50 mm) was added to the beaker which was then placed onto a multi-way magnetic stirrer set at 300 rpm and was left to stir for a minimum of four hours. The rotor head was thoroughly cleaned with acetone and dried. The emulsification process was repeated for the simvastatin loaded batches.

Once, DCM evaporation was complete (four hours), the beakers were removed from the magnetic stirrer, the particles were then washed through a 0.2 micron filter with a 2 litres of distilled water. All of the particles were carefully removed from the filter unit by trituration using a Pasteur pipette in a small volume of water.

The samples were freeze dried for 24 hours (new freeze drier) and stored vacuum packed in the freezer. The samples were then analysed using the Coulter LS230 for size distribution.

PLGA/8% PEG (400 Da) melt-blended particles (sterile) were sieved to a 100-200 micron size fraction prior to use. CMC was used at a concentration of 0.5% w/v in PBS (medium viscosity grade used).

The dissolution media was 0.5% SDS in 0.01 M sodium phosphate adjusted to PH7.

### Method:

### Study 1—Initial Study to Assess Simvastatin Release

The scaffolds were prepared as follows:

3×50 mg 1% simvastatin loaded MPs added to 3×200 mg PLGA/PEG MPs in a weighboat (250 mg total).

1×50 mg blank MPs added to 1×200 mg PLGA/PEG MPs in a weighboat (250 mg total).

To each weighboat 200 ul of CMC was added and mixed in to form a paste (CMC:particle ratio 0.8:1).

Each paste sample was used to fill a 6×12 mm mould. The scaffolds were sintered for 1 hour at 37° C.

The following release conditions were set up in 15 ml Falcon tubes:

3×simvastatin loaded scaffolds and 1×blank loaded scaffold (prepared as above).

3×50 mg simvastatin loaded MPs mixed with 200 mg PLGA/PEG MPs and 1×50 mg blank MPs mixed with 200 mg PLGA/PEG MPs.

3×50 mg simvastatin loaded MPs alone and 1×50 mg blank MPs alone.

5 ml of dissolution media added to each tube and tubes then placed on a 3D rocker at 5 rpm.

At specified time-points the medium was removed from the tubes and replaced with fresh medium. The samples were stored in the fridge prior to being assessed on the Perkin-Elmer Lambda 25 UV-spectrophotometer and the peak at 238 nm recorded.

Results are shown in FIG. 9, and demonstrate approximately 50% suppression of initial release of Simvastatin when DLPs are entrapped within the scaffold. Physical mix of DLPs and PLGA/PEG particles does not suppress release, showing scaffold setting and entrapment of DLPs is required.

### Example 7

### Release Studies with MPs with Higher Simvastatin Loadings Used at Different Concentrations

The scaffolds were prepared as follows:

3×75 mg 5% simvastatin loaded MPs added to 3×175 mg PLGA/PEG (400 Da) MPs in a weighboat (250 mg total).

1×75 mg blank MPs added to 1×175 mg PLGA/PEG MPs in a weighboat (250 mg total).

3×18.75 mg 20% simvastatin loaded MPs added to 3×231.25 mg PLGA/PEG MPs in a weighboat (250 mg total).

1×18.75 mg blank MPs added to 1×231.25 mg PLGA/PEG MPs in a weighboat (250 mg total).

To each weighboat 200 ul of CMC was added and mixed in to form a paste (CMC:particle ratio 0.8:1).

Each paste sample was used to fill a 6×12 mm mould. The scaffolds were sintered for 1 hour at 37° C.

(NB—75 mg of 5% simvastatin loaded MPs and 18.75 mg of 20% simvastatin loaded MPs have the same mass of simvastatin assuming similar entrapment efficiencies).

3 ml of PBS was added to each tube and tubes then placed on a 3D rocker at 5 rpm. Samples harvested for assay as in Example 6 with 3 ml PBS being replaced at each time point.

Results are shown in FIG. 10.

1. An injectable agent delivery system comprising a composition that comprises:
(i) an agent for sustained delivery located within discrete particles; and
(ii) an injectable scaffold material comprising discrete particles which are capable of interacting to form a scaffold.

2. An injectable agent delivery system of claim 1 wherein the agent for sustained delivery is located within the discrete particles which are capable of interacting to form a scaffold.

3. An injectable agent delivery system of claim 1 or claim 2 wherein the agent for sustained delivery is located within discrete particles which are not those capable of interacting to form a scaffold.

4. A composition comprising:
(i) an agent for sustained delivery located within discrete particles; and
(ii) an injectable scaffold material comprising discrete particles which are capable of interacting to form a scaffold
for use in a method of treatment of the human or animal body by surgery or therapy or in a diagnostic method practised on the human or animal body.

5. A composition of claim 4, for pharmaceutical use or cosmetic surgery.

6. A composition comprising:
(i) an agent for sustained delivery located within discrete particles; and
(ii) an injectable scaffold material comprising discrete particles which are capable of interacting to form a scaffold
for use in a method of treatment or prevention of a condition selected from: neurodegeneration disorders (e.g. post stroke, Huntington's, Alzheimer's disease, Parkinson's disease), bone-related disorders (including osteoarthritis, spinal disk atrophy, bone cavities requiring filling, bone fractures requiring regeneration or repair), burns, cancers, liver disorders (including hepatic atrophy), kidney disorders (including atrophy of the kidney), disorders of the bladder, ureter or urethra (including damaged ureter or damaged bladder requiring reconstruction, prolapse of the bladder or the uterus), diabetes mellitus, infertility requiring IVF treatment, muscle wasting disorders (including muscular dystrophy), cardiac disorders (e.g. damaged cardiac tissue post myocardial infarction, congestive heart disease), eye disorders (e.g. damaged or diseased cornea), damaged vasculature requiring regeneration or repair, ulcers, and damaged tissue requiring regeneration or reconstruction (including damaged organ requiring regeneration or reconstruction, and damaged nerves requiring regeneration or reconstruction).

7. A method of treating a subject, such as a mammalian organism, to obtain a desired local physiological or pharmacological effect comprising: administering an injectable agent delivery system according to any of claims 1 to 3 to a site in the subject.

8. A composition of claim 6 or a method of claim 7, wherein the method of treatment or prevention involves controlled release of the agent for delivery to the subject in need of treatment, preferably wherein the initial burst release of the drug is suppressed to provide improved uniformity of release.

9. A composition or method of claim 8, wherein the agent release is sustained for at least 12 hours.

10. A composition of claim 8 or claim 9, wherein the controlled release involves a substantially zero or first order release rate of the agent.

11. An injectable agent delivery system, composition or method of any preceding claim wherein the agent for sustained delivery is a therapeutically, prophylactically or diagnostically active substance.

12. An injectable agent delivery system, composition or method of claim 11 wherein the agent comprises a drug, such as a statin or NSAID, a cell, such as an animal cell, or a signalling molecule, such as a growth factor.

13. An injectable agent delivery system, composition or method of claim 12 wherein the agent comprises simvastatin, atorvastatin, fluvastatin, pravastatin or rosuvastatin.

14. An injectable agent delivery system, composition or method of claim 13 for use in the treatment of orthopaedic indications, craniomaxillofacial surgery or dentistry, for example in dental bone repair such as dental ridge restoration, repair of non-union fractures, or spinal fusion.

15. An injectable agent delivery system, composition or method of claim 11 wherein the agent comprises one or more product selected from: amino acids, peptides, proteins, sugars, antibodies, nucleic acids, antibiotics, antimycotics, growth factors, nutrients, enzymes, hormones, steroids, synthetic materials, adhesion molecules, colourants/dyes, radioisotopes, small molecules, or combinations thereof.

16. An injectable agent delivery system, composition or method of claim 11 wherein the agent comprises one or more cell product selected from: bone cells, osteoprogenitor cells, cartilage cells, muscle cells, liver cells, kidney cells, skin cells, endothelial cells, gut cells, intestinal cells, cardiovascular cells, cardiomycote cells, chondrocytes cells, pulmonary cells, placental cells, amnionic cells, chorionic cells, foetal cells and stem cells.

17. An injectable agent delivery system, composition or method of claim 11 wherein the agent comprises one or more product selected from: epidermal growth factor, platelet derived growth factor, basic fibroblast growth factor, vascular endothelial growth factor, insulin-like growth factor, nerve growth factor, hepatocyte growth factor, transforming growth factors, bone morphogenic proteins, including recombinant human bone morphogenetic protein-2, cytokines including interferons, interleukins, monocyte chemotactic protein-1 (MCP-1), oestrogen, testosterone, kinases, chemokinases, sugars, including glucose, amino acids, calcification factors, amines including dopamine, amine-rich oligopeptides, such as heparin binding domains found in adhesion proteins such as fibronectin and laminin, tamoxifen, cis-platin, peptides and toxoids.

18. An injectable agent delivery system, composition or method of any preceding claim wherein the discrete particles are in a carrier, and wherein the carrier contains one or more suspending agent and/or one or more plasticiser and/or one or more delivery enhancing agent.

19. An injectable agent delivery system, composition or method of any one of the preceding claims wherein the injectable scaffold material comprising discrete particles is capable of solidifying or self-assembling to form a scaffold on or after injection into a subject.

20. An injectable agent delivery system, composition or method of any one of the preceding claims wherein the scaffold that can be formed from the injectable scaffold material comprising discrete particles is porous.

21. An injectable agent delivery system, composition or method of claim 20 wherein the scaffold has pores in the nanometer to millimeter range.

22. An injectable agent delivery system, composition or method of claim 20 or claim 21 wherein the scaffold has about 30% or more pore volume.

23. An injectable agent delivery system, composition or method of any one of claims 20 to 22 wherein some or all of the pores in the scaffold are formed by the gaps which are left between the particles used to form the scaffold during scaffold formation.

24. An injectable agent delivery system, composition or method of any one of the preceding claims wherein solidification of the injectable scaffold material comprising discrete particles into a scaffold is triggered by a change in temperature, a change in pH, a change in mechanical force, or the introduction of a cross-linking agent, setting agent, gelling agent or catalyst.

25. An injectable agent delivery system, composition or method of claim 24 wherein the injectable scaffold material comprising discrete particles is capable of spontaneously solidifying when subjected to an increase in the temperature from room temperature to body temperature.

26. An injectable agent delivery system, composition or method of any one of the preceding claims wherein the discrete particles are capable of cross linking, such that the particles become physically connected and are held together.

27. An injectable agent delivery system, composition or method of any one of the preceding claims wherein the injectable scaffold material comprises discrete particles of one or more polymer.

28. An injectable agent delivery system, composition or method of claim 27 wherein the particles comprise one or more polymer selected from the group comprising: poly (α-hydroxyacids), polyethylene glycol (PEG), polyesters, poly (ε-caprolactone), poly (3-hydroxy-butyrate), poly (s-caproic acid), poly (p-dioxanone), poly (propylene fumarate), poly (ortho esters), polyol/diketene acetal addition polymers, polyanhydrides, poly (sebacic anhydride) (PSA), poly (carboxybiscarboxyphenoxyphosphazene) (PCPP), poly [bis (p-carboxyphenoxy) methane] (PCPM), copolymers of SA, CPP and CPM, poly (amino acids), poly (pseudo amino acids), polyphosphazenes, derivatives of poly [(dichloro) phosphazene], poly [(organo) phosphazenes], polyphosphates, polyethylene glycol polypropylene block co-polymers, and natural polymers such as silk, elastin, chitin, chitosan, fibrin, fibrinogen, polysaccharides, including pectins, alginates, collagen, peptides, polypeptides or proteins, copolymers prepared from the monomers of any two or more of these polymers, random blends of any of two or more of these polymers, and mixtures or combinations thereof.

29. An injectable agent delivery system, composition or method of claim 28 wherein the particles comprise polymer selected from the group comprising poly(α-hydroxyacids), such as poly lactic acid (PLA), polyglycolic acid (PGA), poly(D, L-lactide-co-glycolide)(PLGA), poly D, L-lactic acid (PDLLA), poly-lactide poly-glycolide copolymers, and combinations thereof.

30. An injectable agent delivery system, composition or method of claim 29 wherein the particles comprise polymer which is a blend of a poly(α-hydroxyacid) with poly(ethylene glycol) (PEG), such as a blend of (i) a polymer or copolymer based on glycolic acid and/or lactic acid with (ii) PEG.

31. An injectable agent delivery system, composition or method of any one of claims 27 to 30 wherein the injectable scaffold material comprises particles which are formed of a polymer or a polymer blend that has a glass transition temperature (Tg) from about 25° C. to 50° C., e.g. from about 30° C. to 40° C.

32. An injectable agent delivery system, composition or method of any one of the preceding claims wherein the composition comprises from about 20% (w/w) to about 80% (w/w) injectable scaffold material and from about 20% (w/w) to about 80% (w/w) of a carrier.

33. An injectable agent delivery system, composition or method of any one of the preceding claims wherein the composition comprises:
(i) PLGA/PEG particles comprising 5-10% PEG 400 Da, and having a size range of 10-1000 micron;
(ii) between 1-50% of dry weight of drug loaded particles having a size range of 10-1000 micron, wherein the drug is load into the particles at a concentration of 0.1-80% w/w;
(iii) a liquid carrier comprising 0.2-2% CMC in phosphate buffered saline at a ratio of 0.6-1.5:1.

34. An injectable agent delivery system, composition or method of claim 33 wherein the composition comprises PLGA/PEG particles comprising 5-10% PEG 400 Da.

35. An injectable agent delivery system, composition or method of claim 33 or claim 34 wherein the composition comprises PLGA/PEG particles having a size range of 10-500 micron.

36. An injectable agent delivery system, composition or method of any of claims 33 to 35 wherein the drug loaded particles are PLGA.

37. An injectable agent delivery system, composition or method of any of claims 33 to 36 wherein the drug loaded particles have a size range of 10-200 micron.

38. An injectable agent delivery system, composition or method of any of claims 33 to 37 wherein the drug loaded particles are included at a concentration of 5-30% w/w.

39. An injectable agent delivery system, composition or method of any of claims 33 to 38 wherein the drug is loaded into the particles at a concentration of 0.5-30% w/w.

40. An injectable agent delivery system, composition or method of any of claims 33 to 39 wherein the liquid carrier comprises CMC at 0.5-2.0%.

41. An injectable agent delivery system, composition or method of any of claims 33 to 40 wherein the CMC in phosphate buffered saline is at a ratio of 0.5-1:1.

42. An injectable agent delivery system, composition or method of any of claims 33 to 41 wherein the CMC in phosphate buffered saline is substituted with 1% pluronics F127, 0.5% CMC, and 1-20%, preferably 5%, ethanol in phosphate buffered saline.

43. An injectable agent delivery system, composition or method of any of claims 33 to 42 wherein the drug is Simvastatin and is loaded in a range of 0.01-1 mg per 0.05-1.5 g of scaffold.

44. An injectable agent delivery system, composition or method of any of claims 33 to 43 wherein the composition provides an initial burst of less than about 25% of the total dose of drug.

45. An injectable agent delivery system, composition or method of any of claims 33 to 44 wherein the composition provides less than 25 μg in the first 24 hrs, and approximately 0.5 μg per day thereafter.

46. An injectable agent delivery system, composition or method of any of claims 33 to 45 wherein drug is released for at least 5 days, and preferably for at least one, two or three weeks, most preferably for at least 4-8 weeks or longer.

47. An injectable agent delivery system, composition or method of any one of the preceding claims wherein a scaffold can be formed from the injectable scaffold material without the generation of heat or loss of an organic solvent.

48. A method of forming a scaffold comprising:
(i) providing a product as defined in any one of the preceding claims; and
(ii) allowing the discrete particles of the scaffold material to solidify or self-assemble to form a scaffold having pores.

49. A method of delivering an agent to a subject comprising:
(a) providing an injectable scaffold material, wherein the agent is located within discrete particles within the scaffold material;
(b) administering the scaffold material to a subject;
(c) allowing the scaffold material to solidify/self-assemble in the subject to form a scaffold;
(d) allowing the agent contained within the scaffold material to be released into the subject at the site of administration.

50. The method of claim 49, wherein the injectable scaffold material and agent are as defined in any one of claims 1 to 47.

51. The method of claim 49 or claim 50 wherein in step d) the agent release is sustained over a period at least 12 hours.

52. The method of any one of claims 49 to 51, wherein the method is practised on tissue in vivo or in vitro.

53. The method of any one of claims 49 to 52, wherein solidification of the scaffold material comprising discrete particles into a scaffold is triggered by a change in temperature, a change in pH, a change in mechanical force, or the introduction of a cross-linking agent, setting agent, gelling agent or catalyst.

54. The method of claim 53 wherein solidification of the scaffold material comprising discrete particles into a scaffold is caused by exposing the particles to a change in temperature, from a temperature that is below their Tg to a higher temperature.

55. A scaffold produced by carrying out the method of any one of claims 49 to 54.

56. A method of producing a composition for an injectable agent delivery system, said method comprising:
(a) adding the agent for delivery to a melt blended thermosetting polymer;
(b) allowing the melt blended thermosetting polymer to cool and set; and
(c) creating particles from the set melt blended thermosetting polymer, for example by milling, die-cutting of extruded polymer, or spheronisation.

57. A method of producing a composition for an injectable agent delivery system, said method comprising:
(a) encapsulating the agent for delivery into particles of a non-thermosetting polymer; and
(b) combining particles resulting from step (a) with thermosetting polymer particles.

58. A method of producing a composition for an injectable agent delivery system, said method comprising:
(a) encapsulating the agent for delivery into particles of a non-thermosetting polymer;
(b) combining particles resulting from step (a) with a thermosetting polymer;
(c) melting the thermosetting polymer to embed the particles from step (a) therein and allowing the combination to set; and
(d) creating particles from the set combination, for example milling, die-cutting of extruded polymer or spheronisation.
